(19) United States
(12) Patent Application Publication
(10) Pub. No.: US 2007/0154573 A1
(43) Pub. Date: Jul. 5, 2007

(54) METHOD OF PREPARING AND USING A COLD EXTRACT FROM THE LEAVES OF NERIUM OLEANDER

(76) Inventors: Juay Jamil Rashan, Amman (JO); Heinz-Herbert Fiebig, Freiburg (DE); Farid Jamil Rashan, Mosul (IQ)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/533,376

(22) Filed: Sep. 20, 2006

(30) Foreign Application Priority Data
Dec. 27, 2005 (EP) 05 028 529.5

(51) Int. Cl. C07J 17/00

(52) U.S. Cl. 424/725; 424/774; 536/6.1

(57) ABSTRACT

A method of preparing and using a sterile non-toxic pyrogen-free cold extract from the leaves of Nerium oleander as a supplementary medication to cancer chemo-, hormon and/or radiotherapy to restore and/or ameliorate the immune system of the patient and/or to decrease side effects and increase the antitumor effects of radiotherapy and chemotherapeutics, particularly when used in combination with taxol, adriamycin, cisplatin, 5-fluoro-uracil, alimta, cyclophosphamide, mitomycin-C, navelbine, taxotere and topotecan, respectively, and its use in the manufacture of a medicament for the treatment of one or more cancers of bladder, kidney, liver, ovary, pancreas, testicle, uterus, and vagina as well as pleuramesotheliomas and Hodgkin's lymphomas.

The present application claims priority under 35 U.S.C. §119(a) to European Application No. 05 028 529.5, filed 27 Dec. 2005, the entirety of which is hereby incorporated by reference.

### FIELD OF INVENTION

The present invention relates to the use of a sterile non-toxic pyrogen-free cold extract from the leaves of Nerium oleander as a supplementary medication to cancer chemo-, hormone- and radiotherapy to restore and/or ameliorate the immune system of the patient and to decrease side effects and increase the antitumor effects of radiotherapy and chemotherapeutics, particularly when used in combination with taxol, adriamycin, cisplatin, 5-fluoro-uracil, alimta, cyclophosphamide, mitomycin-C, navelbine, taxotere and topotecan, respectively, and its use in the manufacture of a medicament for the treatment of cancers of bladder, kidney, liver, ovary, pancreas, testicle, uterus, and vagina as well as pleuramesotheliomas and Hodgkin's lymphomas.

### BACKGROUND OF INVENTION

In Arab folk medicine a variety of plants have been used in powder and decoction for treating a number of diseases including cancers and other cell-proliferative and immune deficient diseases. Among these plants are Peganum harmala, Nerium oleander, Arum spp, Capparis spinosa, Ecballium elatrum etc. However, the popularity of these plants decreased because of their extreme toxicity both in animals and in humans.

NZ 514794 describes a process for producing a sterile water-soluble extract from Nerium oleander containing oleandrin and other digoxin-like glycosides, which is produced by cold extraction in water, for use in the treatment of breast tumors, lung tumors, stomach tumors, colorectal tumors, prostate adenocarcinoma and squamous-cell carcinoma. In addition, there are some publications which describe an extract of plant matter of Nerium oleander which is produced by heat extraction (cf. WO 00/16793, EP 0 398 313 A2, EP 0 246 069 B1, WO 02/102395 and DE 39 15 929 A1).

### SUMMARY OF INVENTION

Thus, it is the main object underlying the present invention to provide a new way for the treatment of cancer or to provide a supplementary medication to cancer chemo-, hormone- and radiotherapy.

The solution to the above technical problem is achieved by providing the embodiments characterized in the claims.

In particular, there is provided the use of a sterile water-soluble non-toxic pyrogen-free cold extract, obtainable by a method of preparing a cold extract from Nerium oleander containing oleandrin and other digoxin-type glycosides comprising the steps of:
(i) soaking a dried powder, preferably about 20 g, obtained from the leaves of Nerium oleander, in a sterile medium selected from distilled water, a water/ethanol mixture, a water/methanol mixture, methanol or ethanol, preferably about 100 ml, for a period of about 1 to 50 hours, preferably about 1 to 20 hours, at a temperature in a range of from 0 to 30° C., preferably 0 to 25° C., more preferably 0 to 20° C.,
(ii) filtering the resultant solution under sterile conditions,
(iii) optionally adjusting the volume of the resultant solution to a predetermined volume, preferably about 30 to 40 ml, followed by:
(iv) optionally further filtration under sterile conditions, and
(v) optionally spray drying or freeze drying of the filtrate under sterile conditions, as a supplementary medication to cancer chemo-, hormone- and radiotherapy to restore and/or ameliorate the immune system of the patient and to decrease side effects and increase the antitumor effects of radiotherapy and chemotherapeutics, particularly when used in combination with taxol, adriamycin, cisplatin, 5-fluoro-uracil, alimta, cyclophosphamide, mitomycin-C, navelbine, taxotere and topotecan, respectively, and its use in the manufacture of a medicament for the treatment of cancers of bladder, kidney, liver, ovary, pancreas, testicle, uterus, and vagina as well as pleuramesotheliomas and Hodgkin's lymphomas.

### DETAILED DESCRIPTION

In an embodiment of the present invention, said cold extract can be used in the manufacture of a medicament for the treatment of cancers of bladder, kidney, liver, ovary, pancreas, testicle, uterus, and vagina as well as pleuramesotheliomas and Hodgkin's lymphomas, i.e. it can be used as a single medication to treat these specific cancers. In another embodiment of the present invention, said cold extract can be used as a supplementary medication to cancer chemo-, hormone- and radiotherapy to restore and/or ameliorate the immune system of the patient and to decrease side effects and increase the antitumor effects of radiotherapy and chemotherapeutics, particularly when used in combination with taxol, adriamycin, cisplatin, 5-fluoro-uracil, alimta, cyclophosphamide, mitomycin-C, navelbine, taxotere and topotecan, respectively. When used in combination with these commonly known cancer medicaments, no restriction to the kind of cancer to be treated, is given. For example, cancers of bladder, brain, breast, colorectum, head and neck, kidney, liver, lung, ovary, pancreas, pleuramesothelioma, prostate, stomach, testicle, uterus, and vagina as well as Hodgkin's lymphomas, melanomas and sarcomas, can be treated by such a combination therapy.

In the following Tables 1a and 1b hereinbelow, the result of the combination studies of the cold extract (also designated as Breastin) used in accordance with the present invention, with 5-fluoro-uracil, adriamycin, cisplatin, taxol, alimta, cyclophosphamide, mitomycin-C, navelbine, taxotere and topotecan, respectively, is summarized, where

5FU=5-fluoro-uracil

ADR=adriamycin

PLAT=cisplatin

TAXOL=taxol

CYACT=cyclophosphamide

RS035=cold extract according to the present invention (also designated as Breastin)

Accordingly, a pronounced synergism was observed with taxol in 4/6 cell lines, namely the bladder cell line T24, the colon cell line HCT116, the lung line LXF 1121L and the pancreas cell line PANC1. A synergism was also seen in 2/6 cell lines with adriamycin (T24 and HCT116), in 1/6 cell lines with 5-fluoro-uracil (HCT 116) and in 1/6 cell lines with cisplatin (PANC1); cf. Table 1a. In additional test series, a synergism was also seen with alimta, cyclophosphamide, mitomycin-C, navelbine, taxotere and topotecan; cf. Table 1b.

TABLE 1A
Combination of Breastin with 4 standard agents in
6 human tumor cell lines

| | | | IC50 [μg/ml] of standard agent alone or | | | | | | | |
|---|---|---|---|---|---|---|---|---|---|---|
| | Exp. | Breastin | of Breastin in combination with | | | | | | | |
| Cell line | no. | [μg/ml] | 5-FU | | ADR | | Cis-Platin | | TAXOL | |
| T24 | GF649 | — | 0.429 | | 0.058 | | >30 | | 0.010 | |
| (bladder) | GF649 | 0.1 | 0.401 | − | 0.028 | + | >30 | − | 0.012 | − |
| | GF649 | 0.3 | 0.478 | − | 0.029 | + | >30 | − | 0.003 | ++ |
| HCT116 | GF650 | — | 0.200 | | 0.006 | | >30 | | 0.0020 | |
| (colon) | GF650 | 0.3 | 0.165 | − | 0.006 | − | 19.33 | − | 0.0026 | − |
| | GF650 | 1 | 0.053 | ++ | 0.003 | + | >30 | − | 0.0005 | ++ |
| LXF 1121L | GF651 | — | 0.939 | | 0.013 | | >30 | | 0.0018 | |
| (lung) | GF651 | 0.03 | 0.625 | − | 0.008 | − | >30 | − | 0.0009 | + |
| | GF651 | 0.1 | 0.580 | − | 0.010 | − | >30 | − | 0.0009 | + |
| H460 | GF652 | — | 0.157 | | 0.004 | | 5.26 | | 0.0019 | |
| (lung) | GF652 | 0.1 | 0.202 | − | 0.004 | − | >30 | n.e. | 0.0015 | − |
| | GF652 | 0.3 | 0.145 | − | 0.003 | − | >30 | n.e. | 0.0017 | − |
| MAXF | GF653 | — | 0.139 | | 0.007 | | 1.12 | | 0.00044 | |
| 401NL | | | | | | | | | | |
| (breast) | GF653 | 0.1 | 0.300 | − | 0.005 | − | 0.92 | − | 0.0004 | − |
| | GF653 | 0.3 | 0.079 | − | 0.004 | − | 0.88 | − | 0.000 | + |
| PANC1 | GF654 | — | 0.247 | | 0.022 | | 5.48 | | 0.0019 | |
| (pancreas) | GF654 | 0.03 | 0.199 | − | 0.019 | − | 5.48 | − | 0.002 | − |
| | GF654 | 0.1 | 0.185 | − | 0.015 | − | 2.73 | + | 0.0011 | − |
| Total | | | | 1/6 | | 2/6 | | 1/6 | | 4/6 |
| Synergism | | | | | | | | | | |

n.e.: not evaluable
Evaluating synergism:
−, IC70 (combination) >50% of IC70 standard agent alone
+, IC70 (combination) <=50% of standandard agent alone
++, IC70 (combination) <=30% of standard agent alone
+++, IC70 (combination) <=10% of standard agent alone

TABLE 1B
Combination of Breastin with 6 standard agents in 6 human
tumor cell lines

| | Exp. | Breastin | IC50 [μg/ml] of standard agent alone or of Breastin in combination with | | | | | | | | | | | |
|---|---|---|---|---|---|---|---|---|---|---|---|---|---|---|
| Cell line | no. | [μg/ml] | Alimta | | CYACT | | Mitomycin | | Navelbine¹⁾ | | Taxotere¹⁾ | | Topotecan | |
| T24 | GF693 | — | 0.0010 | | 0.320 | | 0.056 | | 0.144 | | 0.163 | | 0.0032 | |
| (bladder) | GF693 | 0.3 | 0.0018 | − | 0.490 | − | 0.056 | − | 0.173 | − | 0.187 | − | 0.0035 | − |
| | GF693 | 1 | 0.0026 | − | 0.586 | − | 0.042 | − | 0.162 | − | 0.123 | − | 0.0041 | − |
| HCT116 | GF681 | — | 0.021 | | 0.515 | | 0.022 | | 0.060 | | 0.035 | | 0.0066 | |
| (colon) | GF681 | 0.3 | 0.016 | − | 0.716 | − | 0.025 | − | 0.066 | − | 0.065 | − | 0.0060 | − |
| | GF681 | 1 | 0.008 | + | 0.069 | ++ | 0.002 | + | 0.043 | − | 0.020 | − | 0.0045 | − |
| LXF 1121L | GF694 | | 0.014 | | 1.90²⁾ | | 0.055 | | 0.0050 | | 0.079 | | 0.0050 | |
| (lung) | GF694 | 0.1 | 0.012 | − | 0.88²⁾ | + | 0.043 | − | 0.0034 | − | 0.017 | ++ | 0.0026 | − |
| | GF694 | 0.3 | 0.028 | − | 1.00²⁾ | − | 0.032 | − | 0.0041 | − | 0.003 | +++ | 0.0027 | − |
| H460 | GF695 | | 0.018 | | 0.495 | | 0.009 | | 0.057 | | 0.059 | | 0.0079 | |
| (lung) | GF695 | 0.3 | 0.016 | − | 0.243 | + | 0.006 | − | 0.051 | − | 0.057 | − | 0.0067 | − |
| MAXF 401NL | GF682 | | >100 | | 0.121 | | 0.048 | | 0.0083 | | 0.024 | | 0.0017 | |
| (breast) | GF682 | 0.3 | 0.035 | +++ | 0.065 | − | 0.032 | − | 0.0055 | − | 0.011 | + | 0.0018 | − |
| | GF682 | 1 | n.e. | | 0.002 | +++ | 0.0003 | +++ | 0.0022 | ++ | 0.004 | ++ | 0.0002 | ++ |
| PANC1 | GF683 | | <100²⁾ | | 0.193 | | 0.228 | | 0.119 | | 0.153 | | 0.0055 | |
| (pancreas) | GF683 | 0.1 | <100²⁾ | − | 0.235 | − | 0.116 | − | 0.128 | − | 0.219 | − | 0.0025 | + |
| | GF683 | 0.3 | <100²⁾ | − | 0.147 | − | 0.092 | + | 0.120 | − | 0.064 | + | 0.0023 | + |

| | | | | | | |
|---|---|---|---|---|---|---|
| Total synergism | 2/6 | 4/6 | 3/6 | 1/6 | 3/6 | 2/6 |

¹⁾IC50 values given in μg/ml
²⁾based on IC70 values
n.e.: not evaluable
Evaluating synergism:
−, IC70 (combination) >50% of IC70 standard agent alone
+, IC70 (combination) <=50% of standard agent alone
++, IC70 (combination) <=30% of standard agent alone
+++, IC70 (combination) <=10% of standard agent alone

The above-mentioned method of preparing a cold extract of Nerium oleander is an easily reproducible one-step process for preparing a sterile water-soluble non-toxic pyrogen-free cold extract from the leaves of Nerium oleander. The water-soluble cold extract of Nerium oleander can be used in the form of a sterile aqueous extract solution (produced by using steps (i) to (iv)) or in the form of a lyophilized (freeze-dried) powder (produced by using steps (i) to (v)) and, if desired, together with a commonly used pharmaceutically acceptable carrier and optionally one or more additives. This cold extract is non-toxic, pyrogen-free and directed to antioncogenic, antiviral and immunostimulating activity.

The method of drying the Nerium oleander leaves is not particularly restricted and it is possible to dry the leaves at room temperature or at slightly elevated temperatures, e.g., by air drying. However, the dried powder to be used in step (i) is preferably produced by immediately subjecting the collected Nerium oleander leaves to a drying treatment at a temperature of 20 to 50° C. Preferably, the collected Nerium oleander leaves are dried at a temperature of about 40° C. in a drying oven for 48 to 96 hours, preferably 72 hours. Subsequently, the dried Nerium oleander leaves are ground in an electrical grinder to obtain a fine powder having a particle size of, e.g., about 1 to 10 μm.

The active ingredients of Nerium oleander are preferably extracted from the leaves of Nerium oleander having a length of 16 to 19 cm which are harvested during a period of from August to December, most preferably in October.

In the above-mentioned method of preparing a cold extract from Nerium oleander, step (i) is preferably conducted at about room temperature and under sterile conditions.

In step (iv) it is preferred to use a filter having a pore size in a range of 0.22 to 0.45 μm, most preferably a Millipore® filtration system having a pore size of 0.45 and/or 0.22 μm. According to a preferred embodiment of the present invention, a Millipore® filtration having a pore size of 0.45 μm is used, followed by a Millipore® filtration having a pore size of 0.22 μm.

The above-mentioned method of preparing a cold extract from Nerium oleander is suited to achieve a sterile extract with endotoxin concentrations of less than 30 units/ml.

The cold extract used in the course of the present invention preferably includes oleandrins, oleandrigenin as major glycosides with trace amounts of digitoxin, gitoxigenin, flavonoids, rutin and triterpenoides, sugar such as xylose, galactose, glucose, mannose and arabinose, and polysaccharides such as D-galacto-uronic acid, and amino acid moieties. For example, in 125 mg of a dry extract according to the present invention 0.23 mg oleandrin and 0.19 mg oleandrigenin are contained. Other glycosides are present in minor amounts.

Usually, wild Nerium oleander plants are harvested in a selected area close to running water.

According to a preferred embodiment of the present invention the extract is obtainable by:
(a) rinsing the ground powder of Nerium oleander in a sterile medium selected from distilled water, a water/ethanol mixture, a water/methanol mixture, methanol or ethanol, (preferably 1/5 w/v), for 4 to 24 hours at a temperature in a range of 0 to 30° C., preferably 0 to 25° C., more preferably 0 to 20° C., and
(b) filtering the obtained aqueous suspension several times and adjusting the pH value to a range of 5.7 to 6.0 under laminar flow conditions using a filter having a pore size in a range of 0.22 to 0.45 μm, preferably a Millipore® filtration system having a pore size of 0.45 μm and/or a Millipore® filtration system 0.22 μm.

Preferably, (c) the filtered suspension of step (b) may be filtered by using a Millipore® filtration system having a pore size of 0.45 μm and, then, a Millipore® filtration system having a pore size of 0.22 μm under laminar flow conditions, wherein (d) the concentration of endotoxins in the suspension of step (c) is tested and adjusted to less than about 30 units/ml and (e) no bacterial growth in the suspension of step (d) is determined after 48 hours of incubation.

The above-mentioned sterile water-soluble cold extract has both in vitro and in vivo biological characteristics, wherein the in vitro characteristics include:
(a) cytotoxic activity against Hela, K₅₆₂, Raji, KB, Hep-2 (IC₅₀ from 1-5 μg/ml) and a moderate cytotoxicity against P₃₈₈, L₁₂₁₀, T₄₇D, RD, A₅₄₉, L₂₀B (IC₅₀ 6-10 μg/ml),
(b) significant anticancer activity against 31 out of 63 human permanent cell lines, established from bladder, liver, ovary, pancreas, testicle and uterus carcinoma as well as sarcomas and further from brain, colon, head and neck, lung, mammary, melanomas, prostate, and renal carcinoma as well as sarcomas (IC₅₀: 0.09 to 1.14 μg/ml),
(c) significant anticancer activity against 24 out of 67 patient derived human tumors established in nude mice and studied in the tumor stem cell assay; activity was observed in bladder, ovarian, pancreas, pleuramesothelioma, and uterus cancers and further also in brain, colon, lung, mammary, prostate, and renal cancers as well as in melanomas and sarcomas,
(d) hematopoetic stem cells of 3 human donors were less sensitive,
(e) a block at the S-phase of the cell cycle at a concentration of 1 μg/ml for K₅₆₂ and for P₃₈₈ and KB at 10 μg/ml,
(f) a significant ribosome-inactivating protein activity (RIP) at concentrations of less than 1 μg/ml,
(g) no mutagenic effects against two bacterial strains: S. typhimurium TA₁₅₃₀ and S. typhimurium TA₁₅₃₇ at concentrations of up to 50 μg/ml,
(h) release of the cytokines IL-6, TNF-α, IL-1b from PBMC's indicating stimulation of macrophages/monocytes, NK-cells and B-lymphocytes,
(i) IFN-production in whole blood in vitro,
(j) very significant anticomplementary effect at concentrations of less than 1 μg/ml in vitro,
(k) immune-modulatory effect to sheep red blood cells and to P-815 mastocytoma cell lines in vitro (IC₅₀: 0.31 and 2.5 μg/ml, respectively),
(l) inhibition of influenza viruses replication A/Texas/77; A/Philippines/81 and B/Hong Kong/79 in vitro with an IC₅₀=0.3 μg/ml,
(m) activity against HIV-1 at concentrations of less than 1 μg/ml and against Poliovirus type I (Sb1),
(n) significant inhibition of the reverse-transcriptase enzyme in vitro (IC₅₀: 4 to 5 μg/ml), and
(o) no antiviral activity against Herpes virus simplex type I (HSV-1), Reovirus (Reo) and Yellow fever virus (YFV) in vitro at concentrations of up to 20 μg/ml.

The in vivo characteristics of said sterile water-soluble cold extract include:
(a) a significant inhibition of tumor growth of B16-Melanoma in BDF₁ mice (T/C=135% at the dose of 0.025 ml per 25 g mouse), and a significant inhibition of tumor growth of Lewis lung carcinoma in DBF₁ mice (T/C=127% and 137% at the doses of 0.025 ml and 0.05 ml respectively),
(b) no activity against leukaemia models P₃₈₈ and L₁₂₁₀ (T/C=109% and 104% respectively for the dose of 0.05 ml),
(c) no cutaneous effects on guinea pig skin and no inflammatory changes in the eyes of rabbits at the dose of 0.5 ml and 0.1 ml respectively,
(d) a LD₅₀ in mice is 0.35 ml/25 g IM and 0.3 ml and 0.33 ml/25 g IP and SC, respectively, with apparent side effects, at high doses, like tachycardia, myorelaxation and motoric incoordination,
(e) no apparent side effects at doses up to 0.1 ml/25 g in single or repeated doses,
(f) a significant increase in leukocytes count (mostly in lymphocytes) at a dose of 0.05 ml, when studied subchronically for 8 weeks in mice; when studied chronically for 6 months in rats, the extract causes no significant difference in the kidney function tests, but significant increase was observed in the AST and ALP enzymes in the treated groups compared to untreated controls,
(g) no effect on body weight, hemoglobin concentration, erythrocyte count and blood indices like MCV, HCH and MCHC subchronically for a period of 8 weeks in mice, and chronically for a period of 6 months in rats,
(h) no gross-abnormalities in the organs of treated groups when studied chronically for 6 months in rats,
(i) significant myorelaxation activity in mice at concentrations of 0.15 to 0.3 ml/25 g, and
(j) a dose-related sedative effect in mice and a depression of all behaviour aspects at 0.3 ml/25 g.

The sterile water-soluble cold extract can be used for intramuscular, oral, rectal, and intravenous administration.

In a further embodiment, the sterile water-soluble non-toxic pyrogen-free extract, obtainable by a method of preparing a cold extract from Nerium oleander containing oleandrin and other digoxin-type glycosides as described above, can also be used in viral, infectious and inflammatory diseases such as hepatitis B, hepatitis C, influenza, and immune deficient disorders like HIV/AIDS.

In still a further embodiment, the sterile water-soluble non-toxic pyrogen-free extract, obtainable by a method of preparing a cold extract from Nerium oleander containing oleandrin and other digoxin-type glycosides as described above, may be used as a food supplement.

From the above-mentioned sterile cold extract of Nerium oleander a medicament or a pharmaceutical composition can be produced with at least one pharmaceutically acceptable carrier. Preferably, the pharmaceutical composition is suited for injection. Alternatively the pharmaceutical composition may be in the form of an oral or rectal formulation or a topical preparation.

Preferably, the pharmaceutical composition is effective in treating the above-mentioned disorders or diseases, e.g. human tumors, wherein 0.2 to 0.4 ml (2 to 4 mg) are injected by intramuscular route on daily basis followed by an oral maintenance therapy.

Alternatively, 2 to 4 mg of the pharmaceutical composition can be injected by intramuscular route once or twice a week.

The pharmaceutical composition may also be in the form of an oral or a rectal formulation and topical preparation, wherein 7 to 10 mg are given daily.

The treatment of Nerium oleander leaves with water, a mixture of water and ethanol or methanol, ethanol or methanol gives a full range of extracts having antitumor, antiviral, immunmodulatory and cardiotonic activities. The extracts, upon evaporation to dryness, provide a solid residue, characterised by a complex physico-chemical fingerprint.

It should be stressed that the cold extract used in accordance with the present invention is really different from the hot extract. Not only the preparation of the cold extract is quite different from that of a respective hot extract, but also the composition is quite different. While the cold extract is obtained by one-step procedure, the hot extract is prepared by many steps that involves heating, etc., as can be taken, for example, from DE 39 15 929 A1. When the two extracts are analysed using different chemical techniques such as HPLC, LC/MS, high resolution LC/MS, LC/MS/MS, and high resolution NMR, it is evident from the scans that:
(1) The cold extract contains more glycosides when compared to the hot extract and this can be attributed to the fact that during boiling the sugars will cleave off and become free, whereas during cold extraction the sugars become connected to the glycosides.
(2) The results of the above analysis also indicated the presence of triglycosides in the cold extract, whereas monoglycosides were detected in the hot extract. This can be proven by the respective high resolution LC/MS chromatograms.

Furthermore, the respective two different preparations show a different antitumor activity. The cold and the hot extract have been compared in 36 human tumor/cell lines. The cold extract (also designated as Breastin) showed a higher antitumor activity and tumor selectivity than the hot extract. The mean inhibitory concentration IC70 was quite lower for Breastin (2.025 μg/ml) versus 12.994 μg/ml for the hot extract, indicating that the cold extract is 6.4 times more active with a more pronounced tumor selectivity. Therefore, it is evident that the different compositions also are responsible for the much higher antitumor activity of the cold extract. Details are shown in Table 2 hereinbelow.

TABLE 2
IN-VITRO ANTITUMOR ACTIVITY OF RS035
(Cold Extract) and RS034 (Hot Extract

| TUMOR/ | | Cold Extract | Hot Extract |
|---|---|---|---|
| PASSAGE | EXP. | IC70 | IC70 |
| NO. | NO. | μg/ml | μg/ml |
| BXF, Bladder | | | |
| 1218L | *(2) | 1.781 | 14.724 |
| T24 | *(2) | 1.029 | 9.486 |
| CNXF, Central Nervous System | | | |
| 498NL | *(2) | 1.773 | 12.986 |
| SF268 | *(2) | 0.251 | 2.619 |
| CXF, Colon | | | |
| HCT116 | *(2) | 1.447 | 12.276 |
| HT29 | *(2) | 1.798 | 16.295 |
| GXF, Gastric | | | |
| 251L | *(2) | 12.570 | 27.425 |
| HNXF, Head& Neck | | | |
| 536L | *(2) | >30.000 | >30.000 |
| LXF, Lung NSCLC | | | |
| 1121L | *(2) | 0.190 | 2.093 |
| 289L | *(2) | 2.027 | 19.886 |
| 526L | *(2) | 1.514 | 12.410 |
| 529L | *(2) | 0.898 | 6.027 |
| 629L | *(2) | 1.973 | 16.139 |
| H460 | *(2) | 0.884 | 6.340 |
| MAXF, Mammary | | | |
| 401NL | *(2) | 1.852 | 19.043 |
| MCF7 | *(2) | 1.594 | 10.048 |
| MEXF, Melanoma | | | |
| 276L | *(2) | 12.373 | 32.571 |
| 394NL | *(2) | 3.302 | 18.185 |
| 462NL | *(2) | 1.729 | 15.944 |
| 514L | *(2) | 2.085 | 21.089 |
| 520L | *(2) | 2.054 | 15.304 |
| OVXF, Ovary | | | |
| 1619L | FG743IT | >30.000 | >30.000 |
| 899L | *(2) | 2.017 | 19.324 |
| OVCAR3 | *(2) | 1.255 | 17.054 |
| PAXF, Pancreas | | | |
| 1657L | *(2) | 2.116 | 16.399 |
| PANC1 | *(2) | 0.440 | 3.806 |
| PRXF, Prostate | | | |
| 22RV1 | *(2) | 1.911 | 15.096 |
| DU145 | *(2) | 0.558 | 2.330 |
| LNCAP | *(2) | 1.792 | 18.549 |
| PC3M | *(2) | 1.623 | 10.781 |
| PXF, Pleuramesothelioma | | | |
| 1752L | *(2) | 78.790 | 73.027 |
| RXF, renal | | | |
| 1781L | *(2) | 1.524 | 6.185 |
| 393NL | *(2) | 1.439 | 14.771 |
| 486L | *(2) | 1.620 | 13.025 |
| 944L | *(2) | 1.449 | 14.549 |
| UXF, Uterus | | | |
| 1138L | *(2) | 1.826 | 15.614 |
| Mean | n = 36 | 2.025 | 12.994 |

Accordingly, it is evident that a Nerium oleander extract which is produced by heat extraction results in a different extract having less activity when used in a pharmaceutical composition compared to a respective cold extract in preclinical cancer models. For example, a cold extract contains more glycosides and flavonoids, but less polysaccharides than a hot extract.

The following examples are given by way of illustration only and are not limiting the scope of the invention. In the following, the cold extract used in accordance with the present invention is also designated as Breastin.

### EXAMPLES

Preparation of a Nerium oleander Extract and its Characterization

A. Preparation of a Nerium oleander Cold Extract:

200 g of sterile dried, finely ground leaves obtained from Nerium oleander are suspended in 1000 ml of distilled water and are stirred at room temperature (i.e., 22-23° C.) under laminar flow conditions. The solution is filtered several times using Whatman no. 1 filter paper and Millipore® filtrations. The volume of the clear, dark brown filtrate is adjusted to 350 ml and the pH value of the solution is adjusted to 5.9. The filtrate is divided into two equal portions. The first portion is aliquoted under laminar flow conditions into sterile vials and some are stored at 4° C. in the refrigerator and the others are left at room temperature conditions. The second portion of the extract is lyophilized (freeze-dried) under sterile conditions and the dry powder is stored in sterile containers.

B. Sterility Tests:

The following tests are applied:
(1) direct swab culture using different media and direct staining smear technique after 48 hours of incubation,
(2) determination of the concentration of bacterial endotoxins in the extracts and the powder using the conventional Limulus Amoebocyte Lysate (LAL) test, and
(3) the pyrogen test of the extract of the lyophilized powder using rabbits.

No bacterial growth is observed after 48 hours of incubation of the extract and the lyophilized powder with different growth media. Furthermore, the concentration of bacterial endotoxins is less than 30 units/ml. However, the pyrogen tests in rabbits show an increase in the temperature at about 2 to 2.30 hours after intravenous administration.

C. Physico-Chemical Characterisation

100 ml of the aqueous extract are evaporated to dryness at 25° C. under vacuum. The solid extract is kept under vacuum in a desiccator overnight. Aliquots from both the solid residue and the lyophilized fraction are used for TLC and sugar and amino acid tests.

1) Cardiac Glycosides:

The presence of glycosides in the room temperature Nerium oleander extract is examined by the following TLC method, which shows positive results and are compared with the standard glycosides.
(i) Plate: Silica gel G, 250 nm thick, activated at 120° C. for 45 min,
(ii) Mobile phase: benzene: ethanol (7:3),
(iii) Location Reagent: p-anisaldehyde reagent prepared by mixing 10 ml of anisaldehyde, 90 ml of ethanol and 10 ml of concentrated H₂SO₄.

Sample: The solid (100 mg) is suspended in 1 ml of a mixture of CHCl₃ and methanol (1:1) with gentle heating on water bath, and is then centrifuged.

The clear pale yellow solution is placed on the silica gel plate. Comparative samples of the standards (0.2 mg/ml) are prepared using the same solvent and 100 μl of the resulting solution is placed onto the plate.

Procedure and Results

The usual procedure for TLC is carried out. After spraying the reagent, the plate is heated in an oven at 100° C. for 4 min. The spots appeared are varied in color from yellow, blue, violet, green and olive green and with the following R_{f} values: 0.16, 0.23, 0.29, 0.40, 0.45, 0.51, 0.55, 0.60, 0.61, 0.66, 0.70, 0.73. The R_{f} values obtained from the standards are as follows:

| Standards | R_{f} Values |
|---|---|
| Oleandrin | 0.69 |
| Oleandrigenin | 0.65 |
| Digitoxin | 0.59 |
| Gitoxigenin | 0.56 |
| Digoxin | 0.51 |
| Stigmasterol | 0.77 |

Therefore, in general, the R_{f} values of the glycosides present in the extract are well comparable with those of the standards.

2) Sugar Test:

A positive test is obtained on using Fehling's solution reagent, as follows: 50 mg of the solid and the lyophilized fractions are boiled with 0.1 N HCl (5 ml) for about 1 hour. The acidic solution is then neutralised by addition of sodium hydroxide (0.5 N). The total volume is adjusted to about 3 ml by heat evaporation. To this is added a solution of Fehling 1 and 2 (3 ml each) and distilled water (18 ml) and the mixture is heated to the boiling point for 10 minutes. The color turns olive-green with the precipitation of a red solid indicating the presence of sugar moiety in both fractions, due to the formation of Cu₂O.

3) Amino Acids Test:

A positive test is obtained for amino acids on using the ninhydrin solution reagent (0.25 g) in acetone (20 ml), as follows:

Filter Paper Method:

The solid and lyophilized fractions are dissolved in a mixture of water and ethanol (1:2) and one drop of the resulting solution is placed onto filter papers and one drop of the reagent was added. The filter papers are dried over a stream of hot air. A violet color immediately appeared indicating the presence of amino acids in both fractions.

Solution Method:

The solid (50 mg) from both fractions is dissolved in 5 ml water and 3 ml of the reagent is added. The reaction mixture is heated with continuous stirring for about 15 minutes. A deep violet color solution is observed from both fractions indicating the presence of amino acid moieties.

4) Polysaccharides

D-galacto-uronic acid is detected.

5) More Detailed Analysis

The following compounds are isolated in a pure form from the cold aqueous extract of Nerium oleander.
1. odoroside H [digitoxigenin digitaloside] Mt=534
2. odoroside A [digitoxigenin diginoside] Mt=518
3. oleandrin [oleandrigenin-oleandroside] Mt=576
4. oleandrigenin sarmentoside Mt=576
5. oleandrigenin diginoside Mt=576
6. neritaloside (oleandrigenin digitaloside) Mt=592
7. oleandrigenin-B-D-glucopyranoside Mt=594
8. 16-androdigitoxigenin-digitaloside Mt=532
9. 16-anhydrodigitoxigenin sarmentoside Mt=516
10. 16-anhydrodigitoxigenin diginoside Mt=516
11. vanderoside Mt=534
12. adynerine Mt=516
13. 3-0-digitalosyl-8, 14-epoxy-3-hydroxy-carda 20 (22)-enolide Mt=532
14. 3-0-diginosyl-8, 14-epoxy-3-hydroxy-carda 16, 20 (22)-dienolide Mt=514
15. 3-0-sarmentosyl-8, 14-epoxy-3-hydroxy-carda-16, 20 (22) dienolide Mt=514 (which is a new compound)
16. 3-0-digitalosyl-8, 14-epoxy-3-hydroxy-carda 16; 20 (22)-dienolide
17. rutin Mt=510 (separated in large quantities by silica gel, more than 63 mg) some blumenol derivatives plus hexose (glucose) and pentose (xylose, apiose, arabinose) are also isolated in the impure forms and such compounds were not detected previously in Nerium oleander.

6) Conclusion

The cold extract of Nerium oleander is investigated using different chemical techniques including TLC, HPLC, column-chromatographic separation techniques, LC/MS, high resolution mass and LC/MS, LC/MS/MS and high resolution H-NMR.

A number of already known compounds in oleander are isolated in a pure form such as oleandrin, oleandrigenin, adynerin, vanderoside, rutin, etc. On the other hand, the new compound “3-0-sarmentosyl-8, 14-epoxy-3-hydroxy-carda-16, 20 (22)-dienolide” having a molecular weight of 514 is isolated. In addition, for the first time the presence of oleandrigenin-B-D-glucopyranoside has been observed, which according to literature was detected in Nerium odorum and adenium obesum but not in Nerium oleander. Finally, the cold extract also contains some blumenol derivatives plus hexose (glucose) and pentose (xylose, apiose, arabinose) and such compounds are not detected before in oleander.

According to the results of antitumor activities performed on these compounds as described above, this activity can be attributed mainly to oleandrin; oleandrigenin; odoroside A, H; vanderoside; adynerine; 16-anhydrodigitoxigenin-diginoside (IC₇₀>1 μg/mL) and to the blumenol derivatives (IC₇₀ 1-3 μg/mL) and also to some extent to the 3-0-sarmentosyl-8, 14-epoxy-3-hydroxy-carda-16, 20 (22) dienolide (IC₇₀ 6.5 μg/mL). Some activity was also detected in the polysaccharide fraction isolated from the oleander (IC₇₀ <10 μg/ml)

Injectable Preparation

Breastin as an injectable preparation contains the substances as analyzed above, in particular: oleandrin, oleandrigenin and digitoxigenin as major compounds and other digoxin-type glycosides, flavonoids, rutin and triterpenoides, simple sugars such as xylose, galactose, glucose, mannose and arabinose, and polysaccharides such as D-galacto-uronic acid, and amino acid moieties.

Oral Preparation

As above plus starch.

Preclinical Pharmacology

A. Anticancer activity in vitro:

1. In human cell lines in vitro

The anticancer activity of an oral preparation comprising the cold extract according to the present invention (designated as Breastin in the following) is investigated as follows:

The anticancer properties of Breastin are tested in 36 human cancer cell lines in a monolayer assay after 4 days incubation time. 5.000 to 10.000 cells are plated per well in 96 wells, Breastin was added 1 day later and left over for 4 days. An equivalent of cell number is determined with the fluorescence dye propidium iodide. Inhibition of cell number under therapy compared to the control is taken for evaluation. Inhibitory concentrations 50, 70 and 90 are calculated. Breastin shows a high anticancer activity in 31 out of 63 human cancer cell lines derived from bladder, kidney, liver, ovary, pancreas, testicle and uterus as well as pleuramesotheliomas, and furthermore derived from breast, colon, lung, prostate and sarcomas. The IC₅₀ ranged between 0.09 and 1.14 μg/ml. Compared to the established anticancer agents like Cisplatin, Carboplatin or 5-Fluorouracil, Breastin shows a higher overall activity and also a marked tumor selectivity.

2. Effect of Breastin in patient derived tumor models studies in the tumor colony assay invitro

Breastin is studied in 67 human tumor models which are established by implanting patient tumors subcutaneously into nude mice. These tumors remain also in serial passage typical properties of the donor tumors including heterogeneity and tumor sensitivity profiles. Breastin is studied in 6 dose levels from 0.3 mg/ml up-to 30 mg/ml. There is a clear dose response relationship. Overall, Breastin is active in 24/67 tumors (36%). Activity is seen in cancers of the bladder, CNS, colon, lung, ovary, prostate, renal and uterus carcinomas as well as in melanomas and pleuramesotheliomas. Resistant are cancers of the pancreas and sarcomas.

B. Antiviral Effects and Immunstimulation:

The antiviral and immunomodulator effects of an oral preparation comprising the cold extract according to the present invention (designated as Breastin in the following) are investigated as follows:

The antiviral mechanism of Breastin is studied extensively and it was found that Breastin is one of the interferon (IFN) inducers—a special group of potential antiviral compounds IFN—inducers represent a special group of potential antiviral inducing activity. These inducers have many requirements, among these are:

(a) a high IFN-inducing activity.

(b) good solubility in aqueous biological fluids.

(c) broad therapeutic margin.

(d) wide range of antimicrobial activity.

It appears that IFN-inducers stimulate the IFN production in different immunological effector cells and organs and that may explain their efficacy in hepatitis B and C, infuenza A and B and HIV treatment.

Breastin has significant antiviral activity against HIV-1, influenza viruses A and B and polio virus type 1 where the IC₅₀ were found to be 0.1, 0.3 and 0.5 μg/ml, whereas no antiviral activity is demonstrated by Breastin on Herpes virus simplex type 1, Reo and yellow fever viruses (IC₅₀ found to be >20 and >100 μg/ml respectively).

Release of cytokines from human PBMCs give an indication which immunological effector cells are being stimulated by Breastin. There is investigated the effect of Breastin on PBMCs of 3 healthy donors and finds a strong release of IL-6, TNF-alpha and IL-1B and some release of Interferon-gamma at dose levels which are not cytotoxic. These cytokines are released mainly from macrophages/monocytes, TNF-alpha and IL-6 from lymphocytes and natural killer cells. Therefore, Breastin demonstrates immune stimulatory effects.

Breastin also demonstrates an immune-modulatory effect as shown from the primary humoral immune response to sheep red blood cells and P-815 mastocytoma cell lines (IC₅₀=0.31 and 2.5 μg/ml respectively). In addition, it is found that Breastin has no effect on P-815 tumor cells using higher doses and this is another confirmatory evidence concerning the immune modulatory role of Breastin. On the other hand, a significant anti-complimentary activity is demonstrated by Breastin (IC₅₀<0.1 μg/ml). Finally, the effect of Breastin on the IFN-production is also studied. All tested concentrations are active in IFN-production in vitro.

The water-soluble sterile cold extract according to the present invention exhibits a new property, in particular a IFN-inducing activity. This cold extract can be used in treating viruses, such as HIV-1, Hepatitis B and C and influenza viruses as well.

Several criteria are adopted to review the activity of Breastin including the physico-chemical properties and a consistent biological activity both in vitro and in vivo.

1. In Vitro Studies

1.a Primary Humoral Immune Response to Sheep Red Blood Cells
Mouse spleen cells (8-10 weeks old) are co-cultured with sheep erythrocytes for 3 days in 1 ml final volume using 24 well plates. The lymphocytes are harvested, washed and plated (1×10⁵ cells) onto soft-agar with fresh antigen. Complement (guinea pig serum) is added after about 60-90 minutes incubation period and incubation is continued for another 60 minutes. Then, the test is evaluated by using a microscope (plaques counting). After 72 hours of incubation, the lymphocytes are sensitized to the antigen when incubated with antigen again, B-lymphocytes secretes specific antibody which binds to the antigen of the secretary lymphocytes. Addition of complement causes lysis of the antibody coated erythrocytes yielding a plaque and each plaque represents a single antibody producing cell. The results show that the IC₅₀ of the extract is 3.1 (μg/ml) indicating the immune-modulatory effect.

2.a One-way Mixed Lymphocyte Reaction (MLR)
Spleen cells obtained from female albino mice (8-10 weeks old) are co-incubated for 5 days with mitomycin C treated spleen cells from some specific female mice (8-10 weeks). The cells induce a proliferative response in albino C spleen cells which can be measured by labeled precursor incorporation into the DNA. Since the stimulator cells are mitomycin C treated, they do not respond to the albino C cells with proliferation but they do retain their antigenicity.

3.a Cytotoxic and Cytostatic Activity Using the P-815 Mastocytoma Cell-Line
A 96-well plate is filled with a tissue culture medium (100 μl/well), the extract is added to the top row in a 25 μl aliquot, mixed, 25 μl was removed and added to the next lower row and this process is repeated until the end of the plate was reached. The last 25 μl are discarded. 100 μl of cell suspension (P-815 mastocytoma cells, about 30000 cells/well) is then added to each well and incubated for 2 days at 37° C. The proliferation of cells is assessed by the use of a cell counter. The plates are centrifuged, the supernatant is discarded and the cells are washed with phosphate-buffer, saline and a 50 μl/well of Triton-x solution is added followed by shaking the plates for 5-10 minutes. The plates are incubated for 60 minutes at 37° C. and the substrate is added and followed by the addition of the buffer and the plates are read at 405 nm. The result shows that the IC₅₀ of the extract is 2.5 μg/μl showing the immune-modulatory effect against the P-815 mastocytoma cell line.

4.a Anti-Complementary Activity
The inhibition of complement activity is determined as described by Shahat et al. Serial and concentrations of the cold sterile extract according to the present invention are prepared. The assay is performed in a v-well microtiter plate. Rabbit complement (C901 virion/serion immunodiagnostic GmbH) and hemolysing anti-sheep erythrocyte serum (C902 virion/serion immunodiagnostic GmbH) are used. 50 μl of the complement solution (diluted 1:50) are added to 50 μl of each sample concentration. After an incubation period at 37° C. for 30 minutes, 50 μl of a suspension of sensitized sheep erythrocytes are added to each well. Hemolysis is observed optically after an incubation at 37° C. for 60 minutes. Controls consist of sensitized sheep erythrocytes incubated in buffer (no hemolysis), with working solution complement (100% hemolysis with 1:2 and 1:3 diluted working solution complement (partial hemolysis). The IC₅₀ values are calculated and found to be <0.1 μg/μl showing a very significant anticomplementary activity.

5.a Efficacy Against Reverse-Transcriptase (RT) Using Phosphonoformate as Inhibitors of RT
The inhibitory effect of the cold extract according to the present invention against the reverse-transcriptase enzyme is also studied using three different concentrations (0.1, 1 and 10 μg/ml) in vitro and compared with the phosphonoformate (PFA) as inhibitors of RT. The extract demonstrates a significant inhibitory concentration (IC₅₀4-6 μg/ml) when compared with PFA (IC₅₀6-20 μg/ml).

6.a Ribosome-Inactivating Protein (RIP) Assay (Stripe and Barbieri)

The extract is also studied for its RIP activity (ribosome-inhibiting protein) and shows a significant RIP (less than 1 μg/ml).

7.a Interferon (IFN)
The effect of Breastin on INF production in whole blood cell culture is studied in vitro using the method according to Wang et al. Different concentrations of Breastin are used on whole blood obtained from healthy donors. The growth of these cells is assayed after 48 h and the activity is determined using the MTT-assay technique. All measurements are performed in duplicate. All tested concentrations of Breastin are active in stimulating the IFN-production in whole blood in vitro.

2. In Vivo Experiments

2.a P-815 Mastocytoma in vivo

Female mice (20-22 g) are inoculated in the first experiment with 2×10⁶ i.p and with 1×10⁵ P-815 tumor cells. In the second experiment the animals are inoculated i.p with 1×10⁶ P-815 tumor cell. The treatment with Breastin commences post tumor inoculation and the survival time is taken as evaluation criteria.

Results

TABLE 3
Effect of Breastin on P-815 mastocytoma in vivo using Sc. Route
[exp. 1]

| Compound | Dose (mg/kg) | Route | Survival time |
|---|---|---|---|
| Control (H₂O) | / | s.c. | 13 days |
| Breastin | 0.1 | s.c. | 14 days |
| Breastin | 1 | s.c. | 14.3 days |

NB: Tumor cells are administered i.p

TABLE 4
Effect of Breastin on P-815 mastocytoma in vivo using i.m route
[exp. 2]

| Compound | Dose (mg/kg) | Route | Survival time |
|---|---|---|---|
| Control (H₂O) | / | i.m | 13 days |
| Breastin | 0.1 | i.m | 14 days |
| Breastin | 1 | i.m | 14.3 days |

NB: Tumor cells are administered i.p

The above preliminary experiments show that Breastin has no effect on P-185 tumor cells.

2.b B16-Melanoma in vivo

Tumor homogenate of B16-Melanoma is implanted subcutanously to BDF₁ male mice. Breastin is injected intraperitoneally for 9 days at two dose levels (0.025 and 0.05 ml per mouse). Breastin shows a significant increase in the life span of the BDF₁ male mice. The results are obtained according to the following:

ILS=(T−C)/C×100

ILC=increase in the life span.

T=average survival time of the treated mice.

C=average survival time of the untreated mice (control group).

The T/C for the dose 0.025 ml/kg is found to be 135%, which means an increase in survival time of 35%, which is significant.

3. Antiviral Activity

The antiviral efficacy of Breastin is in vitro investigated at different concentrations (0.01, 0.1, 1 and 10 μg/ml) and using different tissue-culture-techniques.

3.a Cell Lines

1-Mock-infected MT-4 cell lines are used to support the growth of the HIV-1 virus.

2-Mock-infected Vero and BHK-21 cell lines are used to support the growth of HSV-1, Sb1, Reo and yellow fever viruses.

Compound concentrations (μg/ml) required to reduce the viability of mock-infected MT-4 cells by 50% are determined by the MTT-method.

Compound concentrations (μg/ml) required to reduce the number of mock-infected Vero and BHK-21 cells by 50% are also determined by using the MTT-method.

3.a Viruses
1) Influenza viruses A/Texas/77; A/Philippines/81 and B/Hong Kong/79.
2) Herpes virus simplex type 1 (HSV-1).
3) Poliovirus type 1 (Sb1).
4) Reovirus (Reo).
5) Yellow fever virus (YFV).
6) Human immunodeficiney virus (HIV-1).

Results

The in vitro antiviral activity of the cold extract of Nerium oleander is shown in Tables 5 and 6.

TABLE 5

| Compound | IC₅₀ (MT-4) | EC₅₀ (HIV-1) |
|---|---|---|
| Breastin | 1.0 | 0.1 |
| AZT | >10 | 0.01 |

Compound concentrations (μg/ml) required to achieve 50% protection of MT-4 cells from the HIV-1 induced cytopathogenecity, as determined by the MTT-method.

TABLE 6

| | IC₅₀ | | EC₅₀ | | | |
|---|---|---|---|---|---|---|
| Compound | ¹Vero | ^{1,2}BHK21 | HSV³-1 | Sb³1 | Reo⁴ | YFV⁵ |
| Breastin | 2 | >100 | >2 | 0.5 | >100 | >100 |

¹Compound concentrations (μg/ml) required to reduce the number of mock-infected Vero and BHK21 cells by 50%.
²Compound concentrations (μg/ml) required to reduce the viability of mock-infected BHK-21 monolayers by 50% as determined by the MTT-method.
³Compound concentrations (μg/ml) required to reduce the HSV-1/Sb1 plaque number by 50% Vero monolayer.
⁴Compound concentrations (μg/ml) required to achieve 50% protection of BHK-21 cells from the Reo virus induced pathogenecity as determined by the MTT method.
⁵Compound concentrations (μg/ml) required to reduce the YFV plaque number by 50% in BHK-21 monolayer.
Breastin inhibits both strains of influenza viruses replication by log10 (IC₅₀ 0.3 μg/ml).

Toxicity Studies

1.B. The Acute Toxicity in Mice
The LD₅₀ of Breastin in albino Balb/C mice using the intramuscular route (IM) is found to be 0.35 ml/25 g (14 ml/kg). The LD₅₀ of Breastin using the intraperitoneal (IP) and the subcutaneous (Sc) routes is found to be 0.3 and 0.33 ml/25 g respectively. Some apparent side effects like tachycardia, myorelaxation and motoric incoordination are observed with high does. When Breastin is administered in single or repeated doses using the IM, IP and Sc routes and up to 0.15 ml/25 g (6 ml/kg) in normal mice, no side effects are apparent.

2.B. The Subacute Toxicity in mice

The potential lethal effect of Breastin is investigated subacutely over a period of 8 weeks with an experimental design of 3 groups with 50 animals/group. Groups comprised male and female balb C/mice.

Group 1 receive 0.025 ml daily IP, group II receive 0.05 ml daily IP, whereas group III receive distilled water daily IP and serve as control.

Morbidity and mortality checks are made daily. Body measurements are scheduled at day (0) before injection and then on a weekly basis. A number of animals are sacrificed/week. Complete blood counts (CBC) and liver and kidney profiles are done on each sacrifice. In addition, biopsies are taken from the liver and kidney during each sacrifice for histopathology study.

The following observations are recorded during the entire period of the experiment:
1) Animals generally gain weight (all groups) indicating that Breastin is not anorexic at the doses used.
2) Breastin shows no significant changes in the hemoglobin concentration or in the erythrocytes count in all groups at the doses used.
3) No significant changes are observed in the blood indices (MCV, MCH and HCHC).
4) Breastin leads to an increase in the leukocytes count in the experimental groups. Significant increases in the lymphocyte count are recorded at both dose levels.
5) Breastin causes no liver and renal function abnormalities.
6) Four mortalities are recorded in group II during the last three weeks of treatment.
7) Pathologically, three biopsies from kidney taken from group II at week 8 show some edematous interstitial tissues. Further, three biopsies taken from the liver of group II at week 8 show multiple focal inflammatory cells in filtered mainly around the central vein with histiocytes. However, some of these changes are also observed in the kidneys and liver biopsies obtained from control untreated animals.

3.B. Special Toxicity Tests for Local Tolerance
The effect of Breastin on the eyes of rabbits and on the skin of guinea pigs is studied using standard protocols. Again two dose levels are used in both tests (0.05 and 0.1 ml).
The results of both tests show that Breastin causes neither erythema nor any cutaneous sensitization in the skin of guinea pigs nor any inflammatory changes in the eyes of the tested rabbits as compared to the untreated controls.

Chronic Toxicity Studies IM for 6 Months in Rats

A total of 240 albino rats (120 males and 120 females) are treated for 6 months. The males are approximately 80 days old. The animals are provided with food and water ad libitum. Animals are housed in suspended cages (6/cage) and the temperature is maintained at 18-22° C. Animals are divided into three groups (80 animals/group including the untreated control animals).

Group 1: Consisting of 80 rats (both sexes) and receive the first dose (approximately 1/10 of the LD₅₀ as determined in mice=0.03 ml).

Group 2: Consisting of 80 rats (both sexes) and receive the second dose (approximately half of the LD₅₀ as determined in mice=0.16 ml).

Group 3: Consisting of 80 rats (both sexes) which receive distilled water (untreated/control).

Animals are dosed by intramuscular injections. Morbidity and mortality checks are made once daily. Additional observations on the general health conditions are also made on the day of administration. Body weight measurements are scheduled to take place prior to the treatment with Breastin followed by additional body weight measurements on the day of sacrifice (once a month). Daily observations are made throughout the entire period of the experiment (6 months) for all groups to see if any behavioral changes occur due to the administration of Breastin.

Samples of blood are obtained by heart puncture technique and the following tests are performed prior to the treatment with the extract and then followed on the day of sacrifice (once a month). These are:

a) Total red blood cells count (RBC).

b) Hemoglobin concentration (HGB).

c) Total white blood cell count (WBC).

d) Differential white blood cell count.

e) Thrombocyte count.

Liver Parameters:

f) Aspartate transaminase (AST).

g) Alanine transaminase (ALT).

h) Alkaline phosphates (ALP).

i) Total albumin (TA).

Kidney Parameters:

j) Urea.

k) Creatinine (Crea).

Results and Discussion
1. All animals remain healthy up to the time of sacrifice. No adverse reactions are noted at the time of observation during the entire period of the experiment. Six mortalities are recorded in group 2, 2 mortalities are recorded in group 1 and only one mortality is recorded in the untreated controls.
2. The gross-anatomy of the organs of the sacrificed animals in all groups is inspected and biopsies are excised from the kidneys and the livers, prior to the treatment with Breastin and then followed on the day of sacrifice (once a month) to look for any gross-abnormalities or histopathological changes. The gross-anatomy of the organs of sacrificed animals looks morphologically normal and no gross-abnormalities are detected.
3. All results are statistically analyzed using different methods of analysis and they indicated:
   a) The statistical analysis performed on the blood cell count throughout the entire period of the experiment (6 months) show that there were no significant differences between all the groups.
   b) The statistical analysis performed on the biochemical results indicate no significant differences in the kidney function tests between the treated groups I and II when compared with the control groups.
   c) Some significant differences are detected in some of the liver function tests between two treated groups I and II. Group II which receives the high dose shows significant increase in both the AST and ALP enzymes compared to group I, whereas no significant differences are detected in the ALT enzyme in both the treated groups.
   d) The histopathological studies performed on biopsies obtained from the kidneys and the livers also give normal results. However, the microscopic anatomy performed on biopsies taken from the liver of the dead animals shows some changes such as infiltration of inflammatory cells and passive congestion, but no central necrosis is detected.

Clinical Studies/Phase I/II-Studies

A. Anticancer Activity of Breastin

Phase I Study

In a phase I study Breastin is tested in 35 patients, 25 presented advanced breast cancers and 10 with colon cancer. Breastin is given IM and escalated to daily 3 to 4 mg (0.4 to 0.6 ml) for 4 months followed by oral administration with 10 to 11 mg (35 to 40 drops per day) which are later adjusted to 15 to 20 drops per day. The limiting toxicity is a rise in body temperature to 38 to 38.2° C.; vomiting, nausea, diarrhea and fatigue, observed in 30% of the patients. There are no treatment related deaths. Tumor regressions and clinical improvements are seen.

Phase II Studies in 383 Patients with Solid Cancers and Hematologic Malignancies

Table 7 summarizes the diagnoses, the number of patients included and the response rate. Patients present the following diagnoses: Breast cancer 150, colorectal 57, prostate 32 and lung 33. In addition 14 patients are treated with stomach, 15 with basal carcinoma of the skin, and less than 10 with melanomas, glioblastomas, osteosarcomas, thyroid, nasopharynx and bladder cancers are included. The response rate is defined by complete and partial responses as well as tumor stabilizations which are seen between 40 and 70%. Part of the patients are pretreated with chemotherapy. Overall, this response rate compares very favourable with other registered agents in the respective tumor entity.

In addition, Breastin is also studied in hematologic malignancies like 13 chronic lymphocytic leukemias (CLL), 10 acute lymphoblastic leukemias, Hodgkin's and non-Hodgkin's lymphomas, and malt lymphoma. The response rate is highest in Hodgkin's lymphoma (43%), in the other diseases up to 20%.

TABLE 7
Summarized anticancer activity of Breastin in phase II studies and late
phase II during 1988 up to date.
Solid cancers and hematologic malignancies

| Types of Cancer | No. of patients | % Response* |
|---|---|---|
| Breast | 150 | 70 |
| Colorectal | 57 | 54 |
| Prostate | 32 | 69 |
| Lung | 33 | 64 |
| Stomach | 14 | 57 |
| Skin basal cell | 15 | 67 |
| Osteosarcoma | 13 | 46 |
| Skin/Melanoma | 4 | 50 |
| Glioblastoma multiforma | 5 | 40 |
| Nasopharynx | 6 | 50 |
| Thyroid | 8 | 63 |
| Larynx/sqamous cell | 3 | 72 |
| Bladder and ureter transitional | 5 | 53 |
| Hodgkin's lymphoma | 7 | 43 |
| Non-hodgkin's lymphoma | 5 | 20 |
| Malt lymphoma | 3 | 33 |
| Chronic lymphocytic leukemia (CLL) | 13 | 8 |
| Acute lymphocytic leukemia (ALL) | 10 | 20 |

*response, complete and partial responses plus stabilisations

Case Report 1: Breast cancer with Secondary Brain Metastasis

A terminal case of breast cancer with a history of right mastectomy. The left breast on examination is free and the left axilla shows painful, hard, multiple mobile lymph nodes (3×4 cm) with no skin involvement. According to a CT-scan of the brain, a small (<1cm) enhancing lesion is seen in the right parietal region in the midline and no evidence of surrounding edema is noted. The lesion is considered to be a metastasis. When the patient presents to the clinic, she is very sick with a huge edema in the right axilla and a loss of vision. She starts the treatment with Breastin using both routes (IM and PO). Three weeks later the patient shows significant changes in the reduction of the right axillary edema and the severity of pains decreased. Five weeks after the treatment with Breastin the patient gains weight (3 kg) and the size of the left axillary lymph nodes show significant reduction in size. The treatment with Breastin is continued for almost one year and during said period the patient is doing well both physically and clinically. A CT-scan performed after one year of treatment with Breastin shows that the size of the lesion in the parietal region and the huge edema in the right axilla is reduced significantly. Unfortunately, the patient died after a surgical operation.

Case Report 2: A Recto-Siqmoid Cancer stage Duke D

A known case of a recto-sigmoid cancer stage Dukes D presents with colicky pains in the lower abdomen and with severe pains at the anal area, etc. She starts the treatment with Breastin using both the IM and PO routes. Two months later a CT-scan is performed and shows that the internal iliac lymph node is getting smaller compared to the previous scan before treatment with Breastin. The CEA at the same time decreases significantly to 4 nanogram (it is much higher before Breastin treatment). Colonoscopy is performed after 4 months of treatment with Breastin and shows that the tumor size inside the lumen is of egg size which is smaller than before. Furthermore, the mass becomes very friable and a regression of the size is noticed. All these findings are accompanied by improvement of the appetite and the disappearance of pains in the abdomen and the perianal area and no more constipation associated with bleeding. The treatment with Breastin continues for about two years during which a careful follow-up is performed using CT-scan, laboratory tests etc. Indeed the patient looks clinically better and significant improvements are achieved and the tumor mass decreases significantly. In addition, the physical performance of the patient is almost normal and almost all the laboratory findings are within the normal limits. A CT-scan is performed after two years of treatment with Breastin which shows a complete regression of the mass from the lumen. The patient continues the maintenance treatment for another two years later.

Case Report 3: Osteogenic Sarcoma

A terminal case of osteogenic sarcoma which is diagnosed by a biopsy from the proximal left tibia. His family refuses to give him either chemotherapy or radiotherapy and they insist that he should be treated with Breastin. He starts the treatment with Breastin using both the IM and PO routes. One month later he shows a bleeding in the tibial region. A bone scan reveals that there is a new periosteal growth and new osteocytes growing in the upper left tibia. The compression at the surroundings is also reduced and the swelling is reduced too. A chest X-ray done at the same time shows no lung metastasis. Six months later another CT-scan is performed of the upper tibial region which reveals a healing process and the swelling in the area is significantly reduced. One year after treatment with Breastin another CT-scan is performed and more osteocytes were observed and more periosteal growth is noted. A chest X-ray is done at the same time and shows no lung metastasis. 14 months later another CT-scan is performed which reveals complete healing and remission to the upper left tibia. Another chest X-ray is performed at the same time and indicates that the lungs are free from any metastasis. The patient goes on for maintenance therapy for another 14 months with Breastin. The patient is still ok after almost 4 years of treatment with Breastin, but dies later when he used other treatments of unknown source.

Case Report 4: Hodgkin's Lymphoma

A 55-years old woman presents with a pain in the left chest, cough and weakness. The clinical examination reveals loss of breathing sound in the left lung. A chest X-ray confirms the presence of fluid in the same lung and a tumor mass. A fine needle aspiration is performed and is diagnosed as malignant lymphoma. The patient refuses to take chemotherapy and decides to take Breastin instead. One month after the treatment with Breastin, an X-ray report shows a significant regression of the tumor mass. Three months later another X-ray is performed and showed that the tumor mass regresses to about half and the physical performance improves remarkably too. Six months later another X-ray scan is performed and shows the complete regression of the mass. However, she has been advised to continue the treatment with Breastin. So she starts the maintenance treatment and continues the treatment for another six months. She has indeed no more complaints and she is physically and psychologically ok. Another X-ray is done after one year and again no mass is demonstrated. A biopsy is taken for pathological study and no pathological findings are noted.

Case Report 5: Glioblastoma multiforma

A 49 years old right-handed man, presents with a right homonomous hemianopia. A CT-scan of the head reveals a mass lesion in the left parietal-occipital region. There is a significant surrounding edema. The patient reports visual changes occurring over a several week interval prior to his evaluation at the hospital. His wife notes increasing difficulty with confusion. The patient reveals on examination a dense right homonomous hemianopia as well as evidence of a parietal-lobe syndrome. The patient is admitted to the hospital to undergo craniotomy with tumor debulking of the brain tumor and biopsy. The results of the fresh frozen section obtained at the time of craniotomy show glioblastoma multiforma belonging to high grade astrocytoma. In addition to craniotomy and radiation therapy, the patient also is treated with chemotherapy. However, the condition of the patient deteriorates and the hospital discharges him. He and his wife decid to take Breastin as the last choice. He starts the treatment with Breastin. However, he is in a very poor medical condition. He is being carried on a stretcher and unable to stand up due to the feebleness occurring in his legs and arms. Two months after treatment with Breastin using both routes, the patient is doing well. The feebleness in his legs and arms as well as his uncomfortable and agitated appearance has almost disappeared. Five months after the treatment with Breastin the patient could walk without any help and a brain CT-scan demonstrated considerable regression of the mass. Another brain CT-scan performed after 10 months reveals “no relapse of the tumor lesion” and the patient is feeling well. Another brain CT-scan performed after 18 months reveals no change with respect to the previous CT-scan. The patient is advised to start a maintenance therapy for another 18 months. Later on, the patient lives normally and he is free from any symptoms.

B. Anti-HIV Activity of Breastin

A total of 14 male and female patients with confirmed HIV/AIDS patients, aged 12-45 years (3 drug users, 6 heterosexual, 4 homosexual and 1 received contaminated blood during surgery) are treated with Breastin, during the period between 1993-2003. These patients can be classified as follows:

3 with class A HIV disease; 6 with class B HIV disease and 5 with a CD⁺4 lymphocyte count <200 μl.

Ten of these cases receive prior treatments (either Retrovir or AZT; antibiotics and some other antiviral agents) in some medical centers before they decide to receive Breastin. The other four patients receive no previous treatment when they are referred to the center. Most of the patients are suffering from the following symptoms:
(a) Continuous weakness and insomnia.
(b) Severe body weight loss and loss of appetite.
(c) Severe diarrhoea.
(d) Presence of opportunistic infections such as pneumonia, Herpes zoster, moniliasis, oral and esophageal candidiasis, etc.
(e) Herpes infection especially at the penis of the males.
(f) Hepatomegally.
(g) Loss of energy.
(h) Depression.

Three routes of administration are followed:
(a) Intramuscular route (IM).
(b) Oral route (PO).
(c) Topical route.

For the intramuscular route, a single daily injection of about 2-4 mg of the active ingredients of Breastin is administered daily for 6 days/week (0.2-0.4 ml). The oral route is used as adjuvant to the IM-route. The oral administration is 10-11 mg in 15 drops/three times daily after meals. The topical route is used only to those patients presenting vascular lesions on the limbs, e.g., Kaposi's sarcoma.

Case Report 1

A 12 year old girl presents in a very poor medical condition:
(a) Suffering from chronic diarrhoea and cough.
(b) Serious loss of the hypodermis adipose.
(c) Severe body weight loss (15 Kg).
(d) Hepatospleenomegally (13 cm×7 cm).
(e) Chest pain during respiration at both mammary region.
(f) Severe fatigue.

The girl has received blood transfusion for several times and the laboratory finding shows that the HIV Antigen and HIV-Antibody tests are positive. The liver enzymes (ALT, AST and the GGT) are above normal values (410, 190 and 420 u/L respectively). She is anemic and the PCV value is less than 26% the total RBC 1500/μl and the WBC is less than 800/μl. The stool examination reveals an infection with Salmonella typhimurium. The girl is first treated with some antibiotic and antifungal drugs for about 2-3 weeks. This is followed later by Breastin treatment using both the IM and the oral routes. Four weeks after the treatment with Breastin she recovers from the oral infection and from pneumonia and no more loss of weight could be observed. The frequency of diarrhea also decrease significantly and both the livers and the spleen show a decrease in size. The laboratory findings indicate a decrease in the ALT, AST and GGT-enzyme activities. Following the result obtained it is decided to continue the treatment with Breastin for another period of time. The treatment continues for another 3 months and the following is observed:
(a) Significant improvements in the general condition of the girl are observed.
(b) Most of the signs noticed before treatment disappear completely (including fatigue, diarrhoea, body weight loss, hepatospleenomegally, etc).
(c) No side effects are noticed.
(d) The viral RNA load is reduced to 50% when compared before treatment.
(e) The Elisa and Western Blott tests show the presence of the virus.

Therefore, it is decided to continue the treatment for another period of time. The patient continues the treatment for another 5 months using the same protocol. The patient's general health condition becomes very good and most of the undesirable symptoms disappear. The laboratory analysis performed on the blood and the patients indicate the followings:
(a) The viral RNA load is reduced to more than 78% when compared before administering the treatment.
(b) Different blood parameters are normal.
(c) The ALT, AST and GGT-enzyme activities readings are normal.
(d) The Elisa and Western Blott tests show the presence the virus.

These results were very promising, however, it is very difficult to interpret the results, that is, the presence of the virus. It can be assumed at this stage that Breastin has a significant effect on the immune system. In other words, Breastin stimulates the immune system of the patient which results in the recovery of the patient. Therefore, it is decided to look at other parameters such as Immunoglobulins IgG, IgA and IgM etc. The patient continues the maintenance therapy for about 9 months. Follow-up studies are performed after the maintenance therapy and the patient is ok clinically and all the laboratory analysis performed are normal.

Case Report 2

A 35 years old homosexual man is diagnosed as HIV-1 positive patient. Immediately after diagnosis he starts the AZT regime treatment. This patient is suffering from severe diarrhea with bloody discharge, cough, severe weight loss, fatigue and he also develops Herpes zoster lesion. However, due to the severe side effects developed by the AZT he discontinues the treatment after 8 weeks. He is referred to the center with complicated side effects such as no appetite with dysphagia mainly for hard food, infection with candida in the eosphagus, severe chest pain especially during swallowing, severe diarrhoea, upper abdominal pain, hair loss, fatigue, no libido and he is in a deep depression. Before he starts the treatment with Breastin, the following tests are formed:
(a) CBC: most of the parameters are normal except that the total WBC was low (1950).
(b) Liver parameters are elevated, especially the ALT, AST and the GGT:

| | |
|---|---|
| ALT | 360 u/l. |
| AST | 140 u/l. |
| GGT | 401 u/l. |

(c) Immunoglobulins:

| | |
|---|---|
| IgG | 2810 mg/dl. |
| IgA | 594 mg/dl. |
| IgM | 930 mg/dl. |

(d) HIV 1 and 2 RNA detection by polymerase chain reaction (PCR) shows that the viral RNA concentration is very high and the patient is very happy with the treatment. The psychological performance changes and he gains weight and hair growth is restored. The patient starts to take Breastin using both routes (IM and the oral routes). Six weeks later the response of the patient to Breastin is remarkable and this is obvious on the general health condition of the patient. Weakness, fatigue, anorexia decrease gradually. Dysphasia, candidal esophageal infection and chest pain disappear. No blood test is performed during this period and the treatment continue for another 12 weeks. The patient is doing well and most of the clinical symptoms disappear.

A blood test is performed after 5 months of treatment with Breastin and revealed:
(a) All blood parameters are normal including the total WBC count.
(b) The liver enzymes level are almost normal.
(c) The immunoglobulins values back to normal level.
(d) The HIV 1- and 2 RNA value decrease significantly to almost 70%.

It is important to point out that most patients treated with the extract according to the present invention show dramatic improvements in the general health and enjoyed sleeping without night sweats and gained weight.

To conclude, it can be noted that Breastin has the following advantages when used to treat HIV/AIDS patients:
(a) Within two months of treatment with Breastin, the total WBC and platelets counts return to normal condition.
(b) Fungal infection in the mouth due to candida and diarrhoea due to Salmonella also disappear within 2-3 months after the treatment.
(c) The loss of body weight and fatigue disappear within 3-4 months after the treatment.
(d) Within about 6-7 months treatment with the extract, herpes infection also disappear completely.
(e) Within 2-4 months treatment the enzymes ALT, AST and ALP decrease to its normal range.
(f) Chronic abscess disappear within 2 months.
(g) The patients show good psychological performance after 2-3 months of treatment with the extract.

C. Anti-HCV Activity of Breastin

During the period between 1996-2000, a total of 10 patients (6 adult males and 4 adult females) aged 39-51 years old are treated with Breastin. Six of these patients receive previously interferon (3 million units per week for a period of 6 months). However, these patients show relapse at the end of the treatment with interferon. The remaining four patients receive no other treatments. The following criteria are followed to enroll patients in this novel preparation:
(a) Confirmed laboratory diagnosis (marked elevation of amino transference enzymes up to 10 folds).
(b) Positive liver biopsy.
(c) Confirmed anti-HCV in the serum.
(d) Clinical symptoms (fever, fatigue, hepatomegally, epistaxis, anorexia etc.).

Case Report 1

A 51 year old woman has been diagnosed as HCV-positive since 1995. She is treated previously with interferon for 6 months, however, she shows a relapse at the end of the treatment. The blood tests performed two months after the treatment with interferon are shown in the table hereinbelow.

Physical Finding

The patient when presented to the clinic is anemic, anorexic, pale, jaundiced, fatigued and she is unable to speak. She starts taking Breastin on March 1996 using the oral route. The initial dose is 10 drops/three times daily after meals. Within four weeks after the treatment, the patient shows rapid improvements in the clinical symptoms such as the disappearance of fever and fatigue and the appetite improved significantly. A blood test performed after 4 weeks treatment with Breastin shows improvement of most liver parameters:

Laboratory Findings of a Patient with HCV treated with Breastin

| | Before | | | After | After | After |
|---|---|---|---|---|---|---|
| Parameter | Breastin | | After 1 month | 3.5 months | 6 months | 10 months |
| ALT | 250 | u/l | 160 | 105 | 41 | 35 |
| AST | 250 | u/l | 145 | 95 | 35 | 22 |
| Albumin | 59 | g/dl | 40 | 29 | 42 | 82 |
| Total bilirubin | 6 | mg/dl | 3.3 | 1.6 | <1 | <1 |
| Direct bilirubin | 3.9 | mg/dl | 2.3 | 1.2 | <1 | <1 |
| Alkaline | 312 | u/l | 270 | 211 | 149 | 140 |
| phosphatase | | | | | | |
| HCV-RNA | 750,000 | u/ml | 750,000 | 410,000 | 170,000 | NEGATIVE |
| (PCR) | | | | | | |

The patient improves significantly and she is impressed by the laboratory results. The dose of Breastin is adjusted to be 15 drops/three times daily. In mid July the patient comes to the clinic for a medical check up. She looks almost healthy and she is energetic and most of the clinical symptoms disappear. A blood test performed after almost three and a half months treatment shows further improvement.

Physical findings show almost normal behavior and are improved significantly compared to the previous period. In mid September she visits the clinic for another medical check up and after almost six months of treatment with Breastin. Indeed she is almost normal physically and clinically.

In January 1997 she has another visit to the clinic for another medical check up and after almost 10 months treatment with Breastin. Physically and clinically the patient is normal. The blood test performed shows normalization of all liver parameters and HCV-RNA determination by PCR is now negative.

A complete disappearance of the anti-HCV after 10 months treatment with Breastin is unexpectedly observed, which demonstrates the activity of Breastin against HCV.

During the period between 2001 to 2005 and due to the significant and encouraging results obtained during the period between 1996-2000, additional clinical studies are performed with Breastin and more than 45 patients (males and females) aged 15-65 years old were enrolled. 40 of these patients are previously treated with different antiviral drugs such as interferon, Amantidine and Ribivirin. However, these patients show relapse at the end of the treatments, five of the patients receive no prior treatments. Again the above criteria are applied to enroll these patients in this herbal treatment. Indeed, almost more than 95% of these patients show physical and clinical improvements following Breastin treatment. Only seven patients are withdrawn from the treatment.

The following conclusions regarding the HCV-patients must be drawn:
(a) Within less than two months of treatment with Breastin most of the liver enzymes improve significantly.
(b) The total and direct bilirubin also improve significantly in most of the patients during the first six weeks of treatment with Breastin.
(c) Disappearance of the abdominal pains and improvement of appetite during the first 6-8 weeks of treatment with Breastin.
(d) Patients are more energetic and they are psychologically improved.
(e) According to the laboratory findings it is noticed that the HCV-RNA (PCR) count declined 80000/month on an average.
(f) The most interesting result obtained on HCV-patients is the disappearance of the anti-HCV from some of the patients after 9-10 months treatment with Breastin which is really a big surprise. Further studies are in progress to elucidate the mechanism of Breastin on HCV-virus and more patients are now enrolled in the treatment.

D. Anti-HBV Activity of Breastin

During the period between 1998-2004, a total of 10 patients, aged 21-54 years old are put on Breastin treatment. Six of these cases are classified as acute and four are classified as chronic HBV. The following criteria are followed to enroll patients in this treatment:
(a) Confirmed anti-HBSAg in the serum.
(b) Marked elevations of the aminotransferase enzymes.
(c) Positive liver biopsy.
(d) Clinical findings such as hepatomegally, ascites, fatigue, anorexia, etc.

Case Report

A 50 year old man was diagnosed in the hospital with acute clinical hepatitis B.

The blood test shows:

HBSAg=8.2 (positive)

HBCAbIGM=positive

HbeAb=negative

He starts the oral treatment with Breastin on the next day. Initially he takes 7-8 mg/daily and after two weeks the dose is increased up to 10-11 mg/daily (10 drops/three times daily). He shows no side effects. The treatment continues for three months. The blood test performed immediately after 3 months shows the following:

HbsAg=negative

HbsAb=negative

Conclusion

Breastin is a herbal non-toxic aqueous solution that contains both polar and non-polar compounds that can stimulate and/or modulate the immune system of the body through enhancing the production of pro-inflammatory cytokines such as IL-2 , IFN- and TNF-α; IL-6 and IL-1B that are able to stimulate the T-cytotoxic lymphocytes, B-lymphocytes, natural killer cells and macrophages to kill virally infected cells either in specific type of recognizing tumor antigens by T-cytotoxic cells or less specific by NK-cells. In addition, Breastin shows no long-term toxicity when studied in animals. Further, it has no mutagenic effects at very high concentration up to 50 μg/ml as indicated when studied on two strains of Salmonella typhimurium TA 1530, TA 1537.

Breastin showed clinical efficacy against Hepatitic B and C, HIV-1, influenza viruses A and B strains.

We claim:

1. A method of preparing and using a sterile water-soluble non-toxic pyrogen-free cold extract from Nerium oleander containing oleandrin and other digoxin-type glycosides comprising the steps of:
(i) soaking a dried powder, obtained from the leaves of Nerium oleander, in a sterile medium selected from distilled water, a water/ethanol mixture, a water/methanol mixture, methanol or ethanol, for a period of 1 to 50 hours at a temperature in a range of from 0 to 30° C.,
(ii) filtering the resultant solution under sterile conditions, and
(vi) administering an effective amount of the cold extract to a patient in need thereof as a supplementary medication to cancer chemo-, hormone- and/or radiotherapy to restore and/or ameliorate the immune system of the patient and/or to increase the antitumor effects of chemotherapeutics and radiotherapy and decrease side effects, respectively.

2. The method according to claim 1, wherein said cold extract is administered as a supplementary medication in cancer therapy in combination with taxol, adriamycin, cisplatin, 5-fluoro-uracil, alimpta, cyclophosphamide, mitomycin-C, navelbine, taxotere or topotecan by enhancing the antitumor activity and reducing side effects and stimulating immunological effector cells.

3. The method according to claim 1, wherein in step (i) active ingredients of Nerium oleander are extracted from the leaves of Nerium oleander having a length of 16 to 19 cm which are harvested during a period of from August to December.

4. The method according to claim 1, wherein step (i) is conducted at room temperature and under sterile conditions.

5. The method according to claim 17, wherein in step (iv) a filter having a pore size in a range of 0.22 to 0.45 μm is used.

6. The method according to claim 1, wherein the sterile extract has endotoxin concentrations of less than 30 units/ml.

7. The method according to claim 1, wherein the extract is obtainable by:
(a) in step (i), rinsing the ground powder of Nerium oleander in a sterile medium selected from distilled water, a water/ethanol mixture, a water/methanol mixture, methanol or ethanol, for 4 to 24 hours at a temperature in a range of 0 to 30° C., and
(b) in step (ii), filtering the obtained aqueous suspension several times and adjusting the pH value to a range of 5.7 to 6.0 under laminar flow conditions using a filter having a pore size in a range of 0.22 to 0.45 μm.

8. The method according to claim 1, wherein said cold extract is administered by intramuscular, oral, rectal and intravenous administration.

9. A method of preparing and using a sterile water-soluble non-toxic pyrogen-free cold extract from Nerium oleander containing oleandrin and other digoxin-type glycosides comprising the steps of:
(i) soaking a dried powder, obtained from the leaves of Nerium oleander, in a sterile medium selected from distilled water, a water/ethanol mixture, a water/methanol mixture, methanol or ethanol, for a period of 1 to 50 hours at a temperature in a range of from 0 to 30° C.,
(ii) filtering the resultant solution under sterile conditions, and
(vi) administering an effective amount of the cold extract to a patient in need thereof for treatment of one or more cancers.

10. The method according to claim 9, wherein in step (i) active ingredients of Nerium oleander are extracted from the leaves of Nerium oleander having a length of 16 to 19 cm which are harvested during a period of from August to December.

11. The method according to claim 9, wherein step (i) is conducted at room temperature and under sterile conditions.

12. The method according to claim 20, wherein in step (iv) a filter having a pore size in a range of 0.22 to 0.45 μm is used.

13. The method according to claim 9, wherein the sterile extract has endotoxin concentrations of less than 30 units/ml.

14. The method according to claim 9, wherein the extract is obtainable by:
(a) in step (i), rinsing the ground powder of Nerium oleander in a sterile medium selected from distilled water, a water/ethanol mixture, a water/methanol mixture, methanol or ethanol, for 4 to 24 hours at a temperature in a range of 0 to 30° C., and
(b) in step (ii), filtering the obtained aqueous suspension several times and adjusting the pH value to a range of 5.7 to 6.0 under laminar flow conditions using a filter having a pore size in a range of 0.22 to 0.45 μm.

15. The method according to claim 9, wherein said cold extract is administered by intramuscular, oral, rectal and intravenous administration.

16. The method according to claim 1, further comprising
(iii) adjusting the volume of the resultant solution to a predetermined volume.

17. The method according to claim 1, further comprising
(iv) further filtration under sterile conditions.

18. The method according to claim 1, further comprising
(v) spray drying or freeze drying of the filtrate under sterile conditions.

19. The method according to claim 9, further comprising
(iii) adjusting the volume of the resultant solution to a predetermined volume.

20. The method according to claim 9, further comprising
(iv) further filtration under sterile conditions.

21. The method according to claim 9, further comprising
(v) spray drying or freeze drying of the filtrate under sterile conditions.

22. The method according to claim 9 wherein the one or more cancers are of bladder, kidney, liver, ovary, pancreas, testicle, uterus, vagina, pleuramesotheliomas and Hodgkin's lymphomas.
